(19) United States
(12) Patent Application Publication
(10) Pub. No.: US 2023/0210463 A1
(43) Pub. Date: Jul. 6, 2023

(54) MONITORING CARDIAC ACTIVITY USING AN IN-EAR DEVICE

(71) Applicant: META PLATFORMS TECHNOLOGIES, LLC, Menlo Park, CA (US)

(72) Inventors: Morteza Khaleghimeybodi, Bothell, WA (US); Srinivas Kota, Redmond, WA (US); Myriam Amsallem, San Bruno, CA (US); Nils Thomas Fritiof Lunner, Redmond, WA (US)

(21) Appl. No.: 17/566,021

(22) Filed: Dec. 30, 2021

(51) Int. Cl. A61B 5/00; A61B 5/024; A61B 5/0245; G02B 27/01

(52) U.S. Cl. CPC A61B 5/6817; A61B 5/02438; A61B 5/0245; G02B 27/017; G02B 2027/0178

(57) ABSTRACT

A system provides ECG monitoring for a user. The system includes an in-ear device and processor. The in-ear device includes in-ear electrodes that capture electrical signals of the pulses of the heartbeat of the user from within the user's ear canal, and an out-of-ear electrode that does not contact any surface of the user's ear or ear canal, and captures electrical signals of pulses of the heartbeat of the user from a fingertip of the user when the user contacts the out-of-ear electrode with their fingertip. The processor generates ECG data using captured electrical signals responsive to a determination that the user has positioned a finger to touch the out-of-ear electrode on the in-ear device. This configuration allows the out-of-ear electrode to, when touched by the user's fingertip, effectively function as an arm electrode, allowing for ECG data to be measured from two different angles, through a path that passes through the user's heart.

### FIELD OF THE INVENTION

This disclosure relates generally to electrocardiography, and more specifically to monitoring cardiac activity (e.g., capturing an electrocardiogram (ECG)) using an in-ear device.

### BACKGROUND

An electrocardiogram (ECG) is a recording of the electrical signal of the heart illustrating how heart's electrical activities (e.g., biopotential) varies over time, and is used in many general health monitoring applications to provide information on a monitored individual's heart rhythm and function. This information can be used to diagnose abnormal activities of the individual's heart, such as atrial fibrillation which may be indicative of stroke risk. Traditionally, ECG data is captured using a medical or hospital-grade electrocardiography monitoring system having a number of electrodes placed on the chest and limbs, allowing for the heart's electrical activities to be monitored from multiple angles. However, such systems are not suitable for frequent monitoring in an out-of-clinic setting. Being able to record ECG data in an out-of-clinic setting would increase the change of diagnosing cardiac rhythm abnormalities, which are often transient and difficult to capture in a clinical environment.

### SUMMARY

Embodiments relate to an in-ear device for measuring ECG data of a user. Some embodiments include a system including an in-ear device and a processor. The in-ear device includes in-ear electrodes configured to capture electrical signals corresponding to the user's heartbeat from within an ear canal of the user, and an out-of-ear electrode configured not to contact a surface of the user's ear when the in-ear device is worn by the user. The processor is configured to determine that a finger of the user is contacting the out-of-ear electrode, and to, capture electrical signals corresponding to the heartbeat of the user at the at least one in-ear electrode and the out-of-ear electrode. The processor is further configured to generate electrocardiogram (ECG) data based upon the captured electrical signals (e.g., based on a voltage difference between the electrical signal captured at the at least one in-ear electrode and the electrical signal captured at the out-of-ear electrode).

Some embodiments include a method for determining an ECG for a user. The method includes, at an in-ear device comprising at least one in-ear electrode configured to contact an inner surface of a user's ear when the in-ear device is worn by the user and an out-of-ear electrode configured to not contact a surface of the user's ear when the in-ear device is worn by the user, determining that a finger of the user is contacting the out-of-ear electrode. Responsive to determining that the user's finger is contacting the out-of-ear electrode, electrical signals corresponding to the heartbeat of the user are captured at the at least one in-ear electrode and the out-of-ear electrode. The method further comprises generating ECG data based upon the captured electrical signals.

Some embodiments include an in-ear device. The in-ear device includes in-ear electrodes, an out-of-ear electrode, and a processor. The in-ear electrodes are configured to capture electrical signals corresponding to a heartbeat of the user from within an ear canal of the user. The out-of-ear electrode is configured to capture electrical signals corresponding to the heartbeat of the user from a fingertip of the user, when the user touches the out-of-ear electrode with their finger. The processor is configured to, responsive to determining that the user's finger is contacting the out-of-ear electrode, capture electrical signals corresponding to the heartbeat of the user between the in-ear electrodes and the out-of-ear electrode, and to generate ECG data based upon the captured electrical signals.

### BRIEF DESCRIPTION OF THE DRAWINGS

FIG. 1 is a block diagram of a ECG monitoring system, in accordance with one or more embodiments.

FIG. 2A is a perspective view of a headset implemented as an eyewear device, in accordance with one or more embodiments.

FIG. 2B is a perspective view of a headset implemented as a head-mounted display, in accordance with one or more embodiments.

FIG. 3 is a diagram of an ECG waveform of a heart in normal sinus rhythm that may be measured by the ECG monitoring system of FIG. 1.

FIG. 4A is a diagram showing how a user can start ECG collection using their hands, in accordance with some embodiments.

FIG. 4B is a diagram showing how a user can start ECG collection using their hands when wearing a headset, in accordance with some embodiments.

FIG. 5 is a diagram showing different monitoring vectors for ECG, in accordance with some embodiments.

FIG. 6 illustrates graphs of example ECG measurements recorded from the left ear, right ear, and chest, in accordance with some embodiments.

FIG. 7 illustrates graphs comparing example ECG measurement between left ear and chest, and between right ear and chest, in accordance with some embodiments.

FIG. 8 illustrates graphs showing examples of heart rate as measured at left ear, right ear, and chest, in accordance with some embodiments.

FIG. 9 illustrates graphs showing example comparisons of heart rate as measure at left ear in comparison to chest, and measured at right ear in comparison to test, in accordance with some embodiments.

FIG. 10 is a flowchart of a method for measuring an ECG of a user, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

A user's heartbeat generates electrical signals that travel across the body. Electrocardiography is the process of measuring these signals to produce an electrocardiogram, or ECG, which can be used to monitor the activity and health of the user's heart. For example, analysis of the user's cardiac activity may be performed to detect cardiac abnormalities. In bipolar ECG collection, electrodes are placed on different areas of the user's body, where the ECG is measured as a voltage difference between the different electrodes. For example, in a conventional ECG measurement system, a pair of electrodes may be paced on the user's left and right arms (referred to as Lead I configuration), on left leg and right arm (Lead II), or on right leg and left arm (Lead III). Each different configuration corresponds to a different viewing vector along which the activity of the heart is measured.

Embodiments relate to an in-ear device for monitoring a user's cardiac health by facilitating measurement of ECGs in an out-of-clinic setting. The in-ear device includes in-ear electrodes that capture electrical signals corresponding to the heartbeat of the user from within an ear canal of the user, and at least one out-of-ear electrode that captures electrical signals corresponding to the heartbeat of the user from a different out-of-ear location on the user's body (e.g., on the user's hand or fingertip). This configuration creates a “loop” through the user's chest, allowing for a clearer ECG signal to be measured in comparison to if only in-ear electrical signals were measured, and allows for the user to perform ECG measurements corresponding to different viewing vectors using the same in-ear device.

In some embodiments, the out-of-ear electrode is located on an outward-facing surface of the in-ear device, such that the out-of-ear electrode does not contact any surface of the user's ear or ear canal. In addition, the out-of-ear electrode is formed such that the user is able to touch the out-of-ear electrode with a different part of their body, such as a fingertip. As such, even though all electrodes are located on the same in-ear device, because the out-of-ear electrode is touched by the user's finger and not by a surface of their ear, the effective bipolar nature of the ECG measurement will correspond to capturing ECG from lead locations in the user's ear and arm, instead of different locations within the user's ear.

A processor, which may be separate from the in-ear device (e.g., in a headset, cuff, or other device) or in the in-ear device, uses the electrical signals to generate ECG data of the user. In some embodiments, the processor is configured to generate ECG data using captured electrical signals responsive to a determination that the user has positioned a finger to touch the out-of-ear electrode on the in-ear device. As such, in some embodiments, the user is able to initiate measurement of ECG data by simply touching their finger to the electrode on their in-ear device, facilitating the capture of ECG data in an out-of-clinic setting, increasing the chances that transient cardiac abnormalities can be detected and diagnosed. Once the ECG data is collected the user can have the options to view it, save it with a time stamp, or even immediately share the ECG data with their physicians to get medical advice and consultations.

Embodiments discussed herein may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to create content in an artificial reality and/or are otherwise used in an artificial reality. The artificial reality system that provides the artificial reality content may be implemented on various platforms, including a wearable device (e.g., headset) connected to a host computer system, a standalone wearable device (e.g., headset), an in-ear device, a mobile device or computing system, or any other hardware platform capable of providing artificial reality content to one or more viewers.

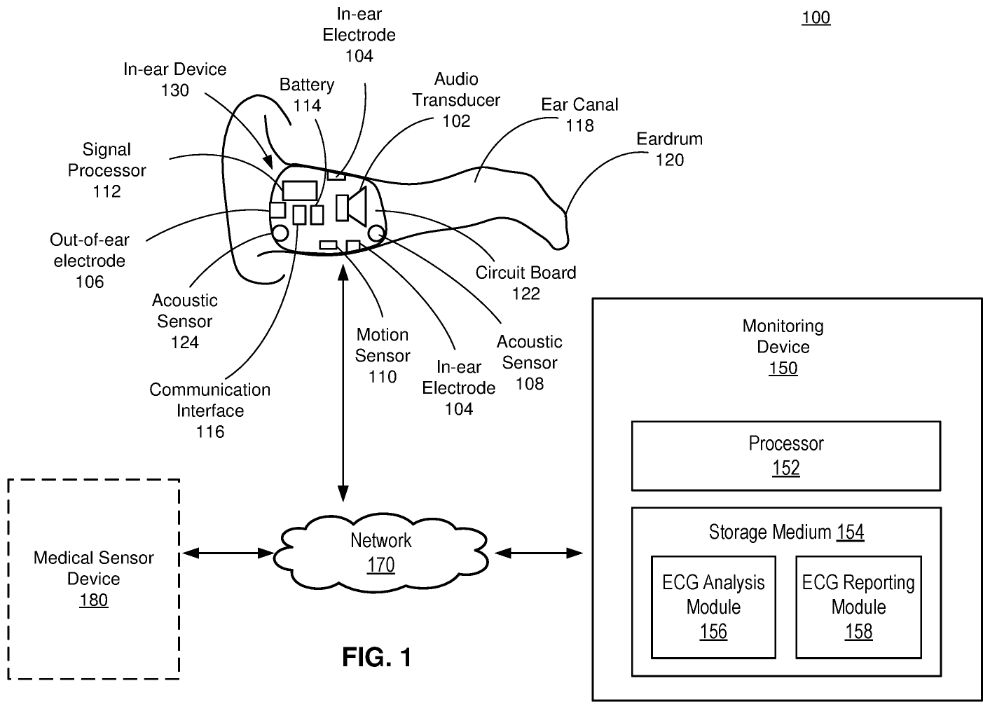

FIG. 1 is a block diagram of an ECG monitoring system 100, in accordance with one or more embodiments. The ECG monitoring system 100 may include an in-ear device 130, a monitoring device 150, a medical sensor device 180, and a network 170. The in-ear device 130 fits within an ear canal 118 of a user near an eardrum 120 and captures various types of data from within the ear canal 118. The monitoring device 150 receives the data from the in-ear device 130 via the network 170 and determines ECG measurements of the user using the data. Some embodiments of the in-ear device 130 and monitoring device 150 have different components than those described here. Similarly, in some cases, functions can be distributed among the components in a different manner than is described here. For example, some or all of the processing for ECG data analysis by the monitoring device 150 as described herein may be performed by the in-ear device 130. In some embodiments, some or all the sensors of the in-ear device 130 may also be in the medical sensor device 180. In some embodiments, the medical sensor device 180 may correspond to a hospital-grade device to be used in a hospital or clinic setting for ECG monitoring, and where the in-ear device 130 is used to measure ECG data when out-of-clinic when the medical sensor device 180 is unavailable.

The in-ear device 130 may include an audio transducer 102, in-ear electrodes 104, an out-of-ear electrode 106, an acoustic sensor 108, a motion sensor 110, a signal processor 112, a battery 114, a communication interface 116, and an acoustic sensor 124. These components of the in-ear device 130 may be mounted to a circuit board 122 that connects the components to each other.

The audio transducer 102 is a speaker that generates sound from audio data and outputs the sound into the ear canal 118. The audio transducer 102 may be used to provide audio messages to the user. For example, the audio transducer 102 may be used to communicate information derived from the user's ECG data (e.g., heart rate, blood pressure levels, etc.) or notify the user when the ECG data of the user indicates a potential health problem or is indicative of abnormal cardiac activity. The audio transducer 102 may also be used to present other types of audio content to the user. In some embodiments, the audio transducer 102 re-broadcasts sound from the local area detected by the acoustic sensor 124, such that the in-ear device 130 provides hear-through functionality even though it is occluding the ear canal 118.

The in-ear electrodes 104 capture electrical signals indicating pulses of the user's heartbeat through the user's ear canal 118. The electrical signals represent the biopotentials created by the pulsation of the user's heart. The electrical signals captured by the in-ear electrodes 104 may be used to generate ECG data defining waveform that represents the electrical activity that is taking place within the heart (e.g., in combination with electrical signals captured by the out-of-ear electrode 106, discussed below).

The in-ear electrodes 104 are positioned at locations on the in-ear device such that they contact a surface of the user's ear canal 118 when the in-ear device 130 is worn by the user. In some embodiments, for better ECG signal quality, the locations of the in-ear electrodes 104 on the in-ear device 130 are selected to contact surfaces of the user's ear canal near the user's arteries when the in-ear device 130 is worn by the user, such as behind the user's tragus.

In some embodiments, the in-ear electrodes 104 are dry electrodes that may be directly in contact with the tissue of the user. A dry electrode does not need gel or some other type of medium or layer between the in-ear electrodes 104 and the tissue. The in-ear electrodes 104 may include hard material electrodes (e.g., including gold-plated brass, iridium oxide, etc.) or soft and/or stretchable material electrodes (e.g., including conductive textiles, conductive polymers, carbon allotropes such as graphene or carbon nanotubes, or poly(3,4-ethylenedioxythiophene) polystyrene sulfonate (PEDOT:PSS).

Although FIG. 1 shows two in-ear electrodes 104 within the in-ear device 130, the in-ear device 130 may include additional or fewer in-ear electrodes to capture the electrical signals. In some embodiments where the in-ear device 130 comprises two or more in-ear electrodes 104, electrical signals measured by each of the two or more in-ear electrodes may be aggregated (e.g., averaged) to generate an overall electrical signal measurement. In some embodiments, multiple in-ear electrodes 104 may be positioned on the in-ear device 130 to contact different locations in the user's ear or ear canal 118. In some embodiments, a plurality of electrodes may be used to contact a larger surface area within the user's ear in comparison to a single in-ear electrode, potentially improving signal quality.

The out-of-ear electrode 106 is located on an outer surface of the in-ear device 130, positioned such that the out-of-ear electrode 106 does not contact any part of the user's ear or ear canal 118 when the in-ear device 130 is worn by the user. In some embodiments, the out-of-ear electrode 106 may also be referred to as a world-facing electrode, as the outer surface of the electrode faces outward away from the user's head, and is exposed to the outside world. The out-of-ear electrode 106 is formed such that when the user holds their hand up near their ear, they are able to touch an outer surface of the out-of-ear electrode 106 with their fingertip. When the user touches the out-of-ear electrode 106 with their fingertip, the out-of-ear electrode 106 captures electrical signals indicating pulses of the user's heartbeat through the user's fingertip, which, combined with the electrical signals captured by the in-ear electrodes 104, may be used to generate ECG data defining waveform that represents the electrical activity that is taking place within the heart, e.g., based upon a voltage difference between the electrical signals captured at the in-ear electrodes and the out-of-ear electrode.

Although FIG. 1 illustrates a single out-of-ear electrode 106, the in-ear device 130 may include additional out-of-ear electrodes to capture the electrical signals. In some embodiments, because the out-of-ear electrode 106 is exposed to the outside world when the user is not touching the out-of-ear electrode 106 with their fingertip, the out-of-ear electrode 106 may be implemented as a dry electrode (e.g., similar to the in-ear electrodes 104).

In some embodiments, the in-ear device contains additional components for capturing other biometric data of the user. The acoustic sensor 108 and motion sensor 110 are examples of sensors for capturing sensor data indicating tissue movements caused by the user's heartbeat, which may be used to generate a waveform representing the tissue movements over time caused by the user's heartbeat. For example, the acoustic sensor 108 captures audio data of sound pressure inside the ear canal 118 caused by the tissue movements, while the motion sensor 110 captures motion data of the tissue movements inside the ear canal 118 of the user caused by the user's heartbeat. In some embodiments, the addition tissue movement data may be used to supplement the generated ECG data. For example, in some embodiments, waveforms representing the tissue movement are compared with ECG data to determine blood pressure levels of the user, using pulse transit time (PTT), i.e., based upon a time interval between a peak in an R wave in the ECG data and a peak in a waveform representing the tissue movement.

The signal processor 112 performs various types of processing to facilitate the capturing of sensor data. For example, as bipolar ECG collection is based upon a voltage difference between the different electrodes, the signal processor 112 includes a differential amplifier to amplify a difference between voltage signals detected at the in-ear electrodes 104 and out-of-ear electrode 106. The signal processor 112 may also include an analog to digital converter (ADC) that converts the electrical signals from the in-ear electrodes 104 and out-of-ear electrode 106 into ECG data. The ADC may also convert the sensor data from other sensors (e.g., the acoustic sensor 108 and/or motion sensor 110) into digital data representing waveforms. The signal processor 112 may perform additional processing of received sensor data (e.g., synchronize the ECG data with the sensor data in time to facilitate determination of blood pressure levels).

The signal processor 112 may also include a digital to analog converter (DAC) that converts digital audio data into analog audio data for rendering by the audio transducer 102. For example, the signal processor 112 may provide audio messages relating the user's ECG data (e.g., indicating abnormal cardiac events) to the audio transducer 102 for rendering to the user.

In some embodiments, the signal processor 112 includes a processing unit to control processing and analysis of received ECG data and/or sensor data. In some embodiments, the signal processor processing unit is further configured to determine when ECG data is collected and processed (e.g., based upon when the out-of-ear electrode 106 detects an electrical signal indicative of being touched by a portion of the user's body). For example, the signal processor may be configured begin measuring of ECG data in response to a detection that the user has contacted the out-of-ear electrode 106 with a portion of their body (e.g., their fingertip), and to continue measuring for at least a threshold amount of time.

The battery 114 provides power to the other components of the in-ear device 130. The battery 114 allows the in-ear device 130 to operate as a mobile device. The battery 114 may be rechargeable via wire or wirelessly.

The communication interface 116 facilitates (e.g., wireless) connection of the in-ear device 130 to other devices, such as the monitoring device 150 via the network 170. For example, the communication interface 116 may transfer data (e.g., ECG data) generated by the in-ear device 130 to the monitoring device 150 for analysis. The in-ear device 130 may also receive biometric data (e.g., analyzed ECG data), audio messages, or other types of information determined from the monitoring device 150 via the communication interface 116 for presentation to the user. In some embodiments, the communication interface 116 includes an antenna and a transceiver.

The medical sensor device 180 is a device that includes one or more sensors used to capture biometric data of the user. The biometric data captured by the medical sensor device 180 may be used in connection with the electrical signals from the in-ear electrodes 104. The medical sensor device 180 may include one or more electrodes, an acoustic sensor 108, motion sensor 110, imaging device, or some combination thereof. The medical sensor device 180 may be a headset, a cuff, a smartphone, a wearable device (e.g., bracelet, watch, etc.), or some other device that can be worn near the skin of the user. In some embodiments, the medical sensor device 180 may correspond to a medical- or hospital-grade electrocardiography monitoring system used for in-clinic ECG monitoring. In some embodiments, the medical sensor device 180 may be used to supplement data generate using the in-ear device 130, and/or calibrate settings of the in-ear device 130 based upon comparisons between ECG data generated using the in-ear device 180 and ECG data measured using the medical sensor device 180.

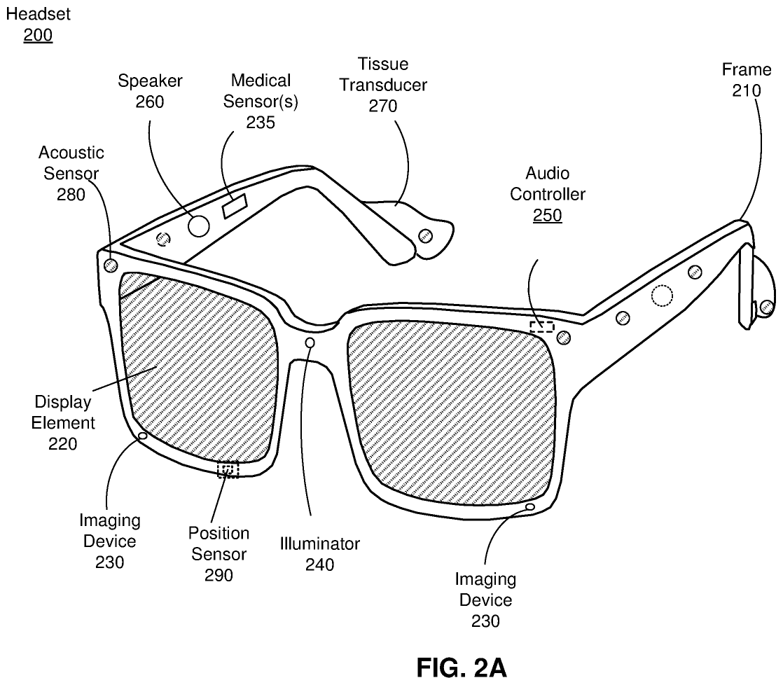

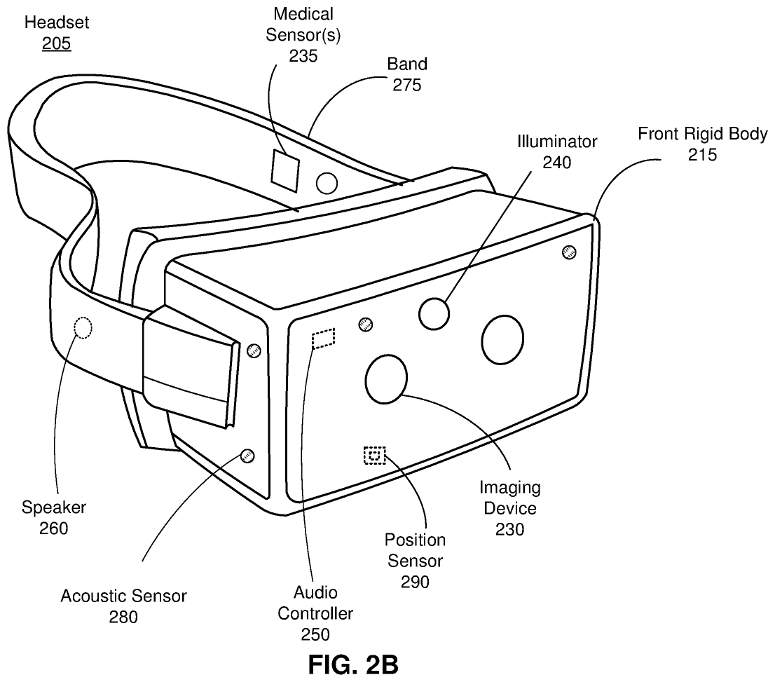

The monitoring device 150 may analyze the ECG data of the user based on the data collected by the in-ear electrodes 104, out-of-ear electrode 106, and/or other sensors (e.g., from the in-ear device 130 and/or medical sensor device 180). In one embodiment, the monitoring device 150 is a headset or head-mounted display (HMD), as discussed in greater detail below in connection with FIGS. 2A and 2B. Alternatively, the monitoring device 150 may be a device having computer functionality, such as a desktop computer, a laptop computer, a personal digital assistant (PDA), a mobile telephone, a smartphone, a tablet, an Internet of Things (IoT) device, a virtual conferencing device, a cuff, or another suitable device.

The monitoring device 150 includes a processor 152 and a storage medium 154. The processor 152 operates in conjunction with the storage medium 154 (e.g., a non-transitory computer-readable storage medium) to carry out various functions attributed to the monitoring device 150 described herein. For example, the storage medium 154 may store one or more modules or applications embodied as instructions executable by the processor 152. The instructions, when executed by the processor 152, cause the processor 152 to carry out the functions attributed to the various modules or applications described herein. The processor 152 may be a single processor or a multi-processor system.

The storage medium 154 includes an ECG analysis module 156 and an ECG reporting module 158. The ECG analysis module 156 performs analysis on received ECG data to determine one or more metrics. For example, in some embodiments, the ECG analysis module 156 may analyze pulses within the generated ECG data to determine a heart rate of the user, analyze the cardiac rhythm of the ECG data to identify potential abnormalities, etc. In some embodiments, the ECG analysis module 156 combines the generated ECG data with other sensor data (e.g., tissue movement data) to determine additional information pertaining to the user (e.g., blood pressure data based on a peak in an R wave in the ECG data and a peak in a waveform representing tissue movement).

In some embodiments, the ECG analysis module 156 uses data from different sensors to determine ECG data over time. For example, in some embodiments, the ECG analysis module 156 receives ECG data measured using the in-ear electrodes 104 and out-of-ear electrode 106 during a first time period, and ECG data measured using the medical sensor device 180 during a second time period. In some embodiments, the ECG analysis module 156 analyzes ECG data generated based upon different measurement configurations, e.g., corresponding to different viewing vectors, such as if the user touches the out-of-ear electrode 106 using a fingertip of their left hand or their right hand, to build an overall ECG profile of the user.

The ECG analysis module 156 analyzes ECG data to generate messages and reports. For example, ECG data may be analyzed to determine user health status, such as whether the user's cardiac activity is indicative of any cardiac abnormalities. In some embodiments, the ECG analysis module 156 analyzes ECG based on biological differences (e.g., gender, age, weight, and BMI, etc.) between different users. The ECG analysis module 156 may monitor the user's ECG history over time and generate real-time information regarding ECG data and health status.

The ECG reporting module 158 communicates ECG data, analysis, and reporting to other devices. For example, an audio message may be provided to the in-ear device 130 for rendering by the audio transducer 102. In another example, the ECG data, analysis, and reporting may be provided to a display of the monitoring device 150. In another example, the ECG reporting module 158 provides the ECG data, analysis, and reporting to a device associated with a physician or other healthcare worker. In some embodiments, ECG reporting module 158 allows the user to opt in to share the history of their measured ECG data with their physicians.

Some or all components of the monitoring device 150 may be located in the in-ear device 130. Similarly, some or all the functionality of the monitoring device 150, ECG determination module 156, and ECG reporting module 158 may be performed by the in-ear device. In some embodiments, the monitoring device 150 is a server connected to the in-ear device 130 via a network 170 that includes the Internet.

The network 170 may include any combination of local area and/or wide area networks, using wired and/or wireless communication systems. In one embodiment, the network 170 uses standard communications technologies and/or protocols. For example, the network 110 includes communication links using technologies such as Ethernet, 802.11 (WiFi), worldwide interoperability for microwave access (WiMAX), 3G, 4G, 5G, code division multiple access (CDMA), digital subscriber line (DSL), BLUETOOTH, Near Field Communication (NFC), Universal Serial Bus (USB), or any combination of protocols. In some embodiments, all or some of the communication links of the network 170 may be encrypted using any suitable technique or techniques.

FIG. 2A is a perspective view of a headset 200 implemented as an eyewear device, in accordance with one or more embodiments. The headset 200 is an example of a monitoring device 150. In some embodiments, the headset 200 is an example of a medical sensor device 180. In some embodiments, the eyewear device is a near eye display (NED). In general, the headset 200 may be worn on the face of a user such that content (e.g., media content) is presented using a display assembly and/or an audio system (e.g., including the in-ear device 130). However, the headset 200 may also be used such that media content is presented to a user in a different manner. Examples of media content presented by the headset 200 include one or more images, video, audio, or some combination thereof. The headset 200 includes a frame, and may include, among other components, a display assembly including one or more display elements 220, a depth camera assembly (DCA), an audio system, and a position sensor 190. While FIG. 2A illustrates the components of the headset 200 in example locations on the headset 200, the components may be located elsewhere on the headset 200, on a peripheral device paired with the headset 200 (e.g., in-ear device 130), or some combination thereof. Similarly, there may be more or fewer components on the headset 200 than what is shown in FIG. 2A.

The frame 210 holds the other components of the headset 200. The frame 210 includes a front part that holds the one or more display elements 220 and end pieces (e.g., temples) to attach to a head of the user. The front part of the frame 210 bridges the top of a nose of the user. The length of the end pieces may be adjustable (e.g., adjustable temple length) to fit different users. The end pieces may also include a portion that curls behind the ear of the user (e.g., temple tip, ear piece).

The frame 210 may include one or more medical sensors 235. In some embodiments, the sensors 235 on the headset 200 may replace or supplement one or more components of the in-ear device 130, such as the in-ear electrodes 104, out-of-ear electrode 106, acoustic sensor 108, and/or motion sensor 110. For example, in one embodiment, the sensors 235 may include a first sensor on the frame 210 at a location near the skin of the user, such as on the temple of the frame 210 or at the user's pinna where the frame 210 rests on the user's ear, having an electrode that contacts the skin of the user that supplements or replaces the in-ear electrode 104, and a second sensor having an out-of-ear world-facing electrode (e.g., located on an outer surface of the frame 210) that supplements or replaces the out-of-ear electrode 106. In some embodiments, the sensors 235 may include different sensors from the ones on the in-ear device 130. For example, the in-ear device 130 may include the in-ear electrode 104 while the headset 205 may include the out-of-ear or world-facing electrode 106.

The one or more display elements 220 provide light to a user wearing the headset 200. As illustrated the headset includes a display element 220 for each eye of a user. In some embodiments, a display element 220 generates image light that is provided to an eyebox of the headset 200. The eyebox is a location in space that an eye of user occupies while wearing the headset 200. For example, a display element 220 may be a waveguide display. A waveguide display includes a light source (e.g., a two-dimensional source, one or more line sources, one or more point sources, etc.) and one or more waveguides. Light from the light source is in-coupled into the one or more waveguides which outputs the light in a manner such that there is pupil replication in an eyebox of the headset 200. In-coupling and/or outcoupling of light from the one or more waveguides may be done using one or more diffraction gratings. In some embodiments, the waveguide display includes a scanning element (e.g., waveguide, mirror, etc.) that scans light from the light source as it is in-coupled into the one or more waveguides. Note that in some embodiments, one or both of the display elements 220 are opaque and do not transmit light from a local area around the headset 200. The local area is the area surrounding the headset 200. For example, the local area may be a room that a user wearing the headset 200 is inside, or the user wearing the headset 200 may be outside and the local area is an outside area. In this context, the headset 200 generates VR content. Alternatively, in some embodiments, one or both of the display elements 220 are at least partially transparent, such that light from the local area may be combined with light from the one or more display elements to produce AR and/or MR content.

In some embodiments, a display element 220 does not generate image light, and instead is a lens that transmits light from the local area to the eyebox. For example, one or both of the display elements 220 may be a lens without correction (non-prescription) or a prescription lens (e.g., single vision, bifocal and trifocal, or progressive) to help correct for defects in a user's eyesight. In some embodiments, the display element 220 may be polarized and/or tinted to protect the user's eyes from the sun.

In some embodiments, the display element 220 may include an additional optics block (not shown). The optics block may include one or more optical elements (e.g., lens, Fresnel lens, etc.) that direct light from the display element 220 to the eyebox. The optics block may, e.g., correct for aberrations in some or all of the image content, magnify some or all of the image, or some combination thereof.

The DCA determines depth information for a portion of a local area surrounding the headset 200. The DCA includes one or more imaging devices 230 and a DCA controller (not shown in FIG. 2A), and may also include an illuminator 240. In some embodiments, the illuminator 240 illuminates a portion of the local area with light. The light may be, e.g., structured light (e.g., dot pattern, bars, etc.) in the infrared (IR), IR flash for time-of-flight, etc. In some embodiments, the one or more imaging devices 130 capture images of the portion of the local area that include the light from the illuminator 240. As illustrated, FIG. 2A shows a single illuminator 240 and two imaging devices 230. In alternate embodiments, there is no illuminator 240 and at least two imaging devices 230.

The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 240), some other technique to determine depth of a scene, or some combination thereof

In some embodiments, the imaging devices 230 may also include one or more image devices to capture image data of the user's eye and/or user's head. For example, the imaging devices 230 may capture image data of the user's eye (e.g., for eye tracking purposes), image data of tissue movements of the user's cheek and/or head (e.g., for determination of blood pressure levels).

The audio system provides audio content. The audio system includes a transducer array, a sensor array, and an audio controller 250. However, in other embodiments, the audio system may include different and/or additional components. Similarly, in some cases, functionality described with reference to the components of the audio system can be distributed among the components in a different manner than is described here. For example, some or all of the functions of the controller may be performed by a remote server.

The transducer array presents sound to user. The transducer array includes a plurality of transducers. A transducer may be a speaker 260 or a tissue transducer 270 (e.g., a bone conduction transducer or a cartilage conduction transducer). Although the speakers 260 are shown exterior to the frame 210, the speakers 260 may be enclosed in the frame 210. In some embodiments, instead of individual speakers for each ear, the headset 200 includes a speaker array comprising multiple speakers integrated into the frame 210 to improve directionality of presented audio content. The tissue transducer 270 couples to the head of the user and directly vibrates tissue (e.g., bone or cartilage) of the user to generate sound. The number and/or locations of transducers may be different from what is shown in FIG. 2A. In some embodiments, the transducer is in the in-ear device 130, such as the audio transducer 102. The in-ear device 130 may be a part of the headset 200 or may be separate from the headset 200.

The sensor array detects sounds within the local area of the headset 200. The sensor array includes a plurality of acoustic sensors 280. An acoustic sensor 280 captures sounds emitted from one or more sound sources in the local area (e.g., a room). Each acoustic sensor is configured to detect sound and convert the detected sound into an electronic format (analog or digital). The acoustic sensors 280 may be acoustic wave sensors, microphones, sound transducers, or similar sensors that are suitable for detecting sounds. In some embodiments, the acoustic sensor 280 is a component of the in-ear device 130 and located outside of the ear canal, such as the acoustic sensor 124.

In some embodiments, one or more acoustic sensors 280 may be placed in an ear canal of each ear (e.g., as in-ear devices, acting as binaural microphones). In some embodiments, the acoustic sensors 280 may be placed on an exterior surface of the headset 200, placed on an interior surface of the headset 200, separate from the headset 200 (e.g., part of some other device), or some combination thereof. The number and/or locations of acoustic sensors 280 may be different from what is shown in FIG. 2A. For example, the number of acoustic detection locations may be increased to increase the amount of audio information collected and the sensitivity and/or accuracy of the information. The acoustic detection locations may be oriented such that the microphone is able to detect sounds in a wide range of directions surrounding the user wearing the headset 200. In some embodiments, an acoustic sensor 280 is a component of the in-ear device 130 located outside the ear canal, such as the acoustic sensor 104.

The audio controller 250 processes information from the sensor array that describes sounds detected by the sensor array. The audio controller 250 may comprise a processor and a computer-readable storage medium. The audio controller 250 may be configured to generate direction of arrival (DOA) estimates, generate acoustic transfer functions (e.g., array transfer functions and/or head-related transfer functions), track the location of sound sources, form beams in the direction of sound sources, classify sound sources, generate sound filters for the speakers 260, or some combination thereof. In some embodiments, the audio controller 250 performs the functionality discussed herein for the processor 152, such as ECG analysis and/or reporting.

The position sensor 290 generates one or more measurement signals in response to motion of the headset 200. The position sensor 290 may be located on a portion of the frame 210 of the headset 200. The position sensor 290 may include an inertial measurement unit (IMU). Examples of position sensor 290 include: one or more accelerometers, one or more gyroscopes, one or more magnetometers, another suitable type of sensor that detects motion, a type of sensor used for error correction of the IMU, or some combination thereof.

In some embodiments, the headset 200 may provide for simultaneous localization and mapping (SLAM) for a position of the headset 200 and updating of a model of the local area. For example, the headset 200 may include a passive camera assembly (PCA) that generates color image data. The PCA may include one or more RGB cameras that capture images of some or all of the local area. In some embodiments, some or all of the imaging devices 230 of the DCA may also function as the PCA. The images captured by the PCA and the depth information determined by the DCA may be used to determine parameters of the local area, generate a model of the local area, update a model of the local area, or some combination thereof. Furthermore, the position sensor 290 tracks the position (e.g., location and pose) of the headset 200 within the room.

FIG. 2B is a perspective view of a headset 205 implemented as an HMD, in accordance with one or more embodiments. In embodiments that describe an AR system and/or a MR system, portions of a front side of the HMD are at least partially transparent in the visible band (-380 nm to 750 nm), and portions of the HMD that are between the front side of the HMD and an eye of the user are at least partially transparent (e.g., a partially transparent electronic display). The HMD includes a front rigid body 215 and a band 275. The headset 205 includes many of the same components described above with reference to FIG. 2A, but modified to integrate with the HMD form factor. For example, the HMD includes a display assembly, a DCA, an audio system, a position sensor 290, and one or more medical sensors 235. FIG. 2B shows the illuminator 240, a plurality of the speakers 260, a plurality of the imaging devices 230, a plurality of acoustic sensors 280, and the position sensor 290. The speakers 260 may be located in various locations, such as coupled to the band 275 (as shown), coupled to front rigid body 215, or may be configured to be inserted within the ear canal of a user (e.g., with in-ear device 130).

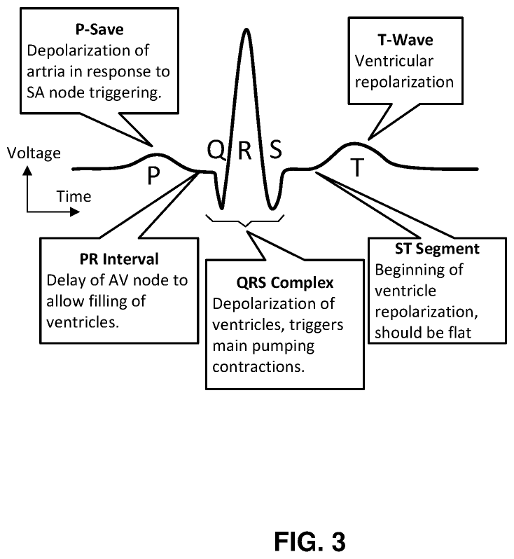

FIG. 3 is a diagram of an ECG waveform of a heart in normal sinus rhythm that may be measured by the ECG monitoring system 100 of FIG. 1. The ECG may include pulses corresponding to the user's heartbeat. Each pulse is made of a plurality of waves, each corresponding to a respective depolarization or repolarization of a portion of the user's heart, as part of the heart's sinus rhythm. For example, as shown in FIG. 3, these waves include a P wave, followed by a QRS complex including a Q wave, an R wave, and an S wave, and followed by a T wave. Each wave corresponds to a depolarization or repolarization of a portion of the user's the heart

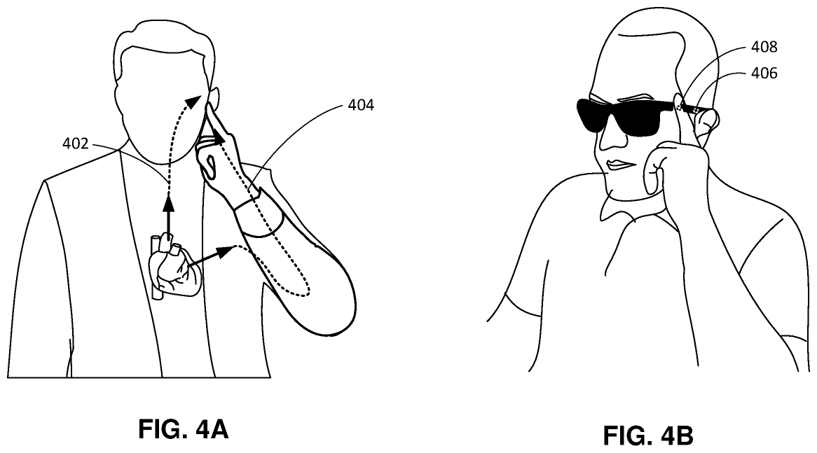

FIG. 4A is a diagram showing how a user can start ECG collection using their hands, and different monitoring vectors for ECG, in accordance with some embodiments. As discussed above, the user may wear an in-ear device (e.g., in-ear device 130) having one or more in-ear electrodes that contact a surface of the user's ear or ear canal, and at least one out-of-ear electrode that does not contact the user's ear tissue. The in-ear device is part of the ECG monitoring system 100.

When the user touches the out-of-ear electrode with their fingertip, e.g., left arm fingertip as shown in FIG. 4A, the in-ear electrode measures a first voltage 402 reflecting electrical activity of the user's heart from the inside of the user's ear canal, while the out-of-ear electrode measures a second voltage 404 reflecting electrical activity of the user's heart from the user's fingertip. The voltage difference as measured by the in-ear electrodes and out-of-ear electrode is used to generate ECG data for the user. In some embodiments, the user may need to hold their fingertip on the out-of-ear electrode for at least a threshold amount of time for the ECG monitoring system to capture sufficient data for an accurate ECG measurement (e.g., at least a threshold number of ECG waveforms). For example, in some embodiments, the threshold amount of time may be 5-6 seconds. In other embodiments, the threshold amount of time may be at least 10 seconds. In some embodiments, the ECG monitoring system may prompt the user to hold their finger for at least the threshold amount of time, or notify the user when the threshold amount of time has been reached, e.g., by playing an audio cue using audio transducer 102.

As shown in FIG. 4A, even though the in-ear electrodes and out-of-ear electrode may all be located on the same in-ear device, by positioning the out-of-ear electrode such that it was not contact any of the user's ear tissue, but instead, is touched by the user's fingertip during measurement, the effective bipolar nature of the ECG collection corresponds to capturing ECG from two distant locations on the user's body (i.e., an ear and a limb). In other words, the out-of-ear electrode, when touched by the user's fingertip, effectively functions as an arm electrode, allowing for ECG data to be measured from two different angles, through a path that passes through the user's heart. This may provide a clearer ECG signal to be measured in comparison to if only in-ear electrical signals were measured, where the electrical path between the electrodes would not pass through the user's heart, but instead be solely within the user's head. In addition, because the ECG signal is measured based upon a voltage difference between the in-ear electrodes and out-of-ear electrode, signal quality is improved when the electrodes are located on different parts of the user's body due to a stronger voltage difference, compared to only using electrodes contacting the user's ear or ear canal.

Although FIG. 4A shows the user touching the out-of-ear electrode of an in-ear device with their same-side fingertip (e.g., touching the out-of-ear electrode of an in-ear device within their left ear using their left arm fingertip), in other embodiments, the user may touch the out-of-ear electrode using an opposite-side fingertip, e.g., right fingertip touching the out-of-ear electrode of an in-ear device located in the user's left ear. In some embodiments, the ECG monitoring system may be a binaural system in which an in-ear device is worn in each ear of the user. The user may simultaneously touch an out-of-ear electrode on each in-ear device using two different fingers to measure two different ECG signals. In some embodiments, the two different ECG signals may have different levels of signal quality, and are used to derive an overall ECG signal for the user (e.g., an average of the two ECG signals). In some embodiments, a time difference between ECG signals on the left and right sides may be used to derive additional information, e.g., generate an estimation of blood pressure. In some embodiments, the ECG monitoring system may prompt the user to use a specific finger to touch the out-of-ear electrode of an in-ear device of a specific ear, e.g., using audio transducer 102.

FIG. 4B is a diagram showing how a user can start ECG collection using their hands when wearing headset, e.g., an eyewear device or an HMD, in accordance with some embodiments. As discussed above, in some embodiments, the headset, such as that illustrated in FIG. 2A or FIG. 2B, may include sensors (e.g., located on the headset frame 210 or band 275) that supplement or replace the in-ear electrode and/or out-of-ear electrode of the in-ear device. For example, the headset may contain a first electrode 406 located on an inner surface of the frame that, when worn by the user, contacts the user's skin (e.g., near the user's temple or pinna), and a second electrode 408 on an outer surface of the frame that does not contact any portion of the user's ear or head. When the user touches the second electrode with their fingertip, the first electrode measures a first voltage reflecting electrical activity of the user's heart from a point on the user's head, while the second electrode measures a second voltage reflecting electrical activity of the user's heart from the user's fingertip, where the voltage difference as measured by the first and second electrodes is used to generate ECG data for the user. It is understood that in some embodiments, different combinations of electrodes may be used to measure ECG data for the user. For example, in some embodiments, ECG may be measured based on an in-ear electrode touching an inner surface of the user's ear canal and an electrode on the outer surface of the headset touched by the user's finger, or based on an electrode on the inner surface of the headset touching the user's skin and an out-of-ear electrode located on an in-ear device touched by the user's finger.

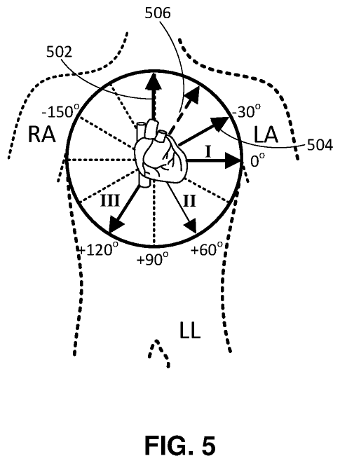

FIG. 5 is a diagram showing different viewing vectors for ECG, in accordance with some embodiments. As discussed above, in a conventional ECG measurement system, pairs of electrodes places on different locations of the user's body may be used to measure ECG data of the user from different angles. Each pair of electrodes is associated with a respective viewing vector, corresponding to an angle at which the ECG data provides a view of the user's heart. For example, as illustrated in FIG. 5, Lead I configuration (corresponding to a pair of electrodes places on the user's left and right arms) may be represented as a horizontal viewing vector, while Leads II and III (corresponding to a first electrode on an arm of the user, and a second electrodes on an opposite-side leg of the user) may corresponding to downward diagonal viewing vectors. When performing ECG in a clinical setting, different configurations are used in order to view activity of the heart from different angles.

When the user holds their finger to their ear to perform in-ear ECG measurement by contacting the out-of-ear electrode of the in-ear device, the in-ear electrode of the in-ear device corresponds to an upward vector 502. On the other hand, the out-of-ear electrode contacting the user's finger corresponds to leftward or rightward-pointing vector 504 (e.g., a vector pointing approximately 30° above the horizontal), depending on which arm was used. The resulting combined vector may correspond to a diagonal vector 506 (e.g., approximately 60° from horizontal) with similar features to the Lead III configuration (or to the Lead II configuration, depending on the arm used). In some embodiments, in a binaural system where the user wears an in-ear device in each ear, when the user simultaneously touches an out-of-ear electrode on each in-ear device using two different fingers, bipolar ECG data may be collected based on a voltage difference between the two out-of-ear electrodes (corresponding to the user's fingertips on each arm), generating ECG data similar to the Lead I configuration. As such, by interacting with the in-ear device in different ways (e.g., using different arms to touch to out-of-ear electrodes), ECG data associated with different viewing vectors can be measured using the in-ear device. Although FIG. 5 illustrates view vectors with specific angles, it is understood that in other embodiments, the angles of the viewing vectors when using the ECG monitoring system may be different from those illustrated in FIG. 5.

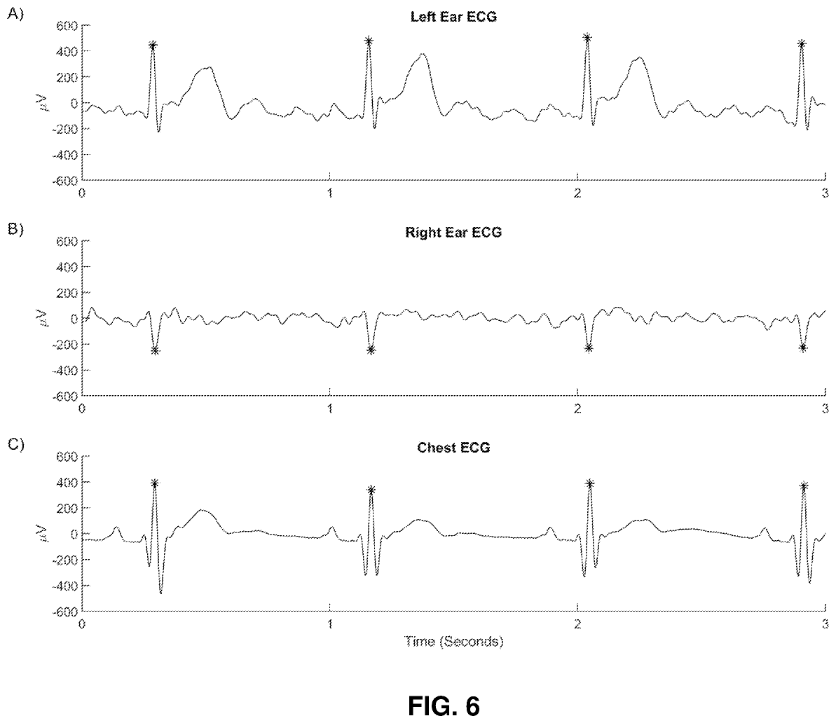

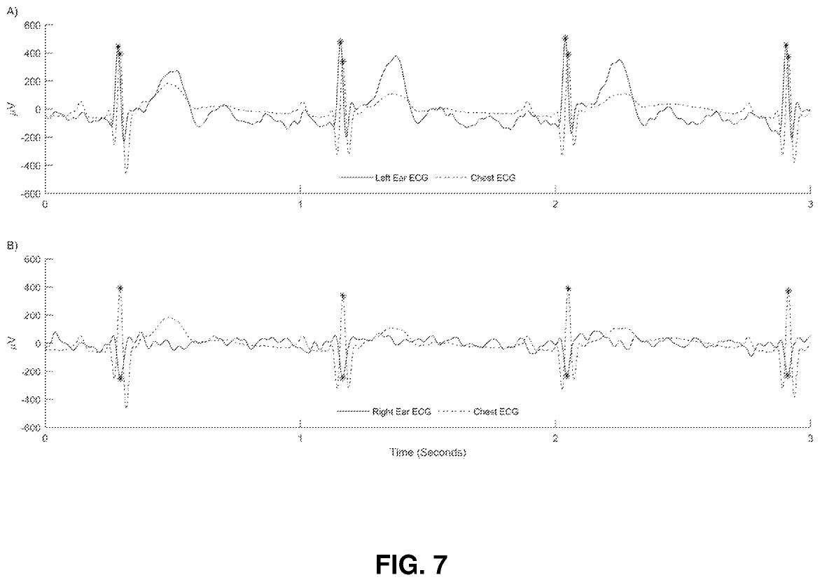

FIGS. 6-7 illustrate graphs of ECG measurements that may be obtained using an in-ear device in comparison to a chest ECG measured in a clinical setting using electrodes placed on the user's chest, in accordance with some embodiments. FIG. 6 illustrates graphs of example ECG measurements recorded using left ear-measured ECG (left ear ECG), right ear-measured ECG (right ear ECG), and chest-measured ECG (chest ECG), in accordance with some embodiments. The ECG measurements shown in the left ear ECG and right ear ECG graphs of FIG. 6 are collected using an ECG monitoring system having an in-ear device, such as the ECG monitoring system 100 illustrated in FIG. 1, based upon measurements taken when the user touches the out-of-ear electrode of the in-ear device using their hand, where left ear ECG refers to ECG measured when the user touches the out-of-ear electrode using their left hand, and right ear ECG refers to ECG measured when the user touches the out-of-ear electrode using their right hand. On the other hand, the ECG measurements shown in the chest ECG graph of FIG. 6 corresponds to ECG data collected using a medical or hospital-grade electrocardiography monitoring system having electrodes placed on the user's chest. The graphs of FIG. 6 illustrate three seconds of ECG measurements, where the R-peaks of each ECG waveform are marked by an asterisk. As shown in FIG. 6, the ECG waveforms produced by measuring left ear ECG and by using chest electrodes substantially match, while the ECG waveforms measured using right ear ECG exhibit opposite polarity compared to the left ear and chest ECG waveforms, due to the different viewing vector of the measurement.

FIG. 7 illustrates graphs comparing example ECG measurements generated using left ear ECG and chest, and generated using right ear ECG and chest, in accordance with some embodiments. For example, FIG. 7 illustrates a first graph in which the left ear ECG measurement shown in FIG. 6 is superimposed on the chest ECG measurement, and a second graph in which the right ear ECG measurement in superimposed on the chest ECG measurement, where the first and second graphs show substantial alignment between the R-peaks of the ECG measurements measured at the ears and chest.

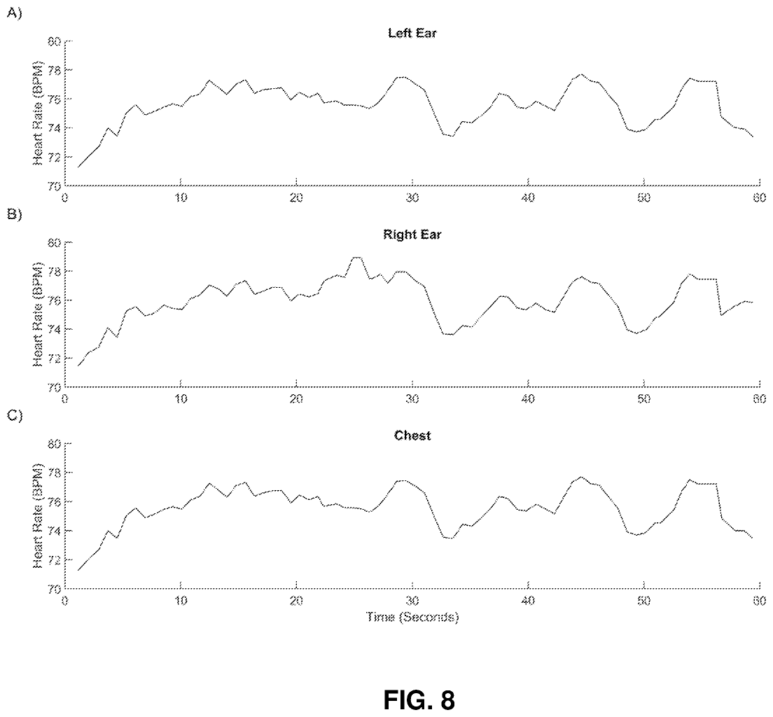

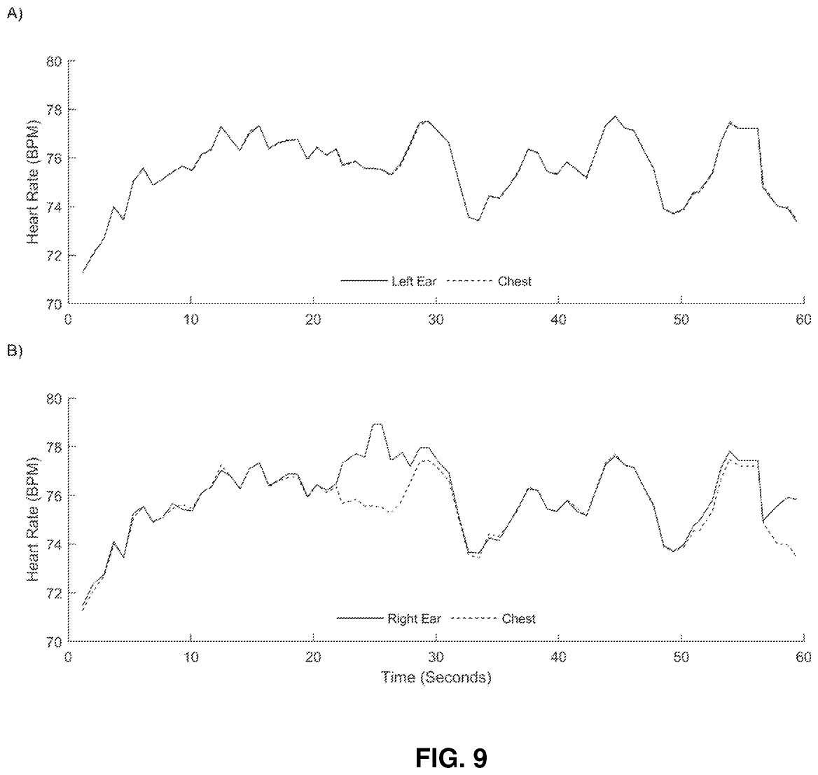

FIGS. 8-9 illustrate graphs of heart rate measurements that may be obtained using an in-ear device in comparison to a chest ECG measured in a clinical setting using electrodes placed on the user's chest, in accordance with some embodiments. As discussed above, in some embodiments, the ECG analysis module may analyze generated ECG data to generate one or more additional metrics, such as analyzing pulses within the generated ECG data to determine a heart rate of the user. FIG. 8 illustrates graphs showing examples of heart rate determined using left ear ECG, right ear ECG, and chest ECG, in accordance with some embodiments. FIG. 9 illustrates graphs showing example comparisons of heart rate as determined using left ear ECG in comparison to chest ECG, and right ear ECG in comparison to chest ECG, in accordance with some embodiments. The heart rate data corresponding to left and right ear ECG shown FIGS. 8-9 is generated based on ECG data collected using an ECG monitoring system having an in-ear device (e.g., ECG monitoring system 100 illustrated in FIG. 1) based upon measurements taken when the user touches the out-of-ear electrode of the in-ear device using their left hand and right hand, respectively. On the other hand, the heart rate data corresponding to chest shown in FIGS. 8-9 corresponds to ECG data collected using a medical or hospital-grade electrocardiography monitoring system having electrodes placed on the user's chest.

As illustrated in FIGS. 8 and 9, the heart rate determined using left ear ECG measurements aligns with that determined using chest ECG measurements, while the heart rate determined using right ear ECG measurements may exhibit some small differences from the chest ECG-based heart rate due to differences in viewing vector (e.g., due to differences in the viewing vector when collecting left-ear ECG data in comparison to chest ECG data), but maintains the same overall heart rate in beats per minute.

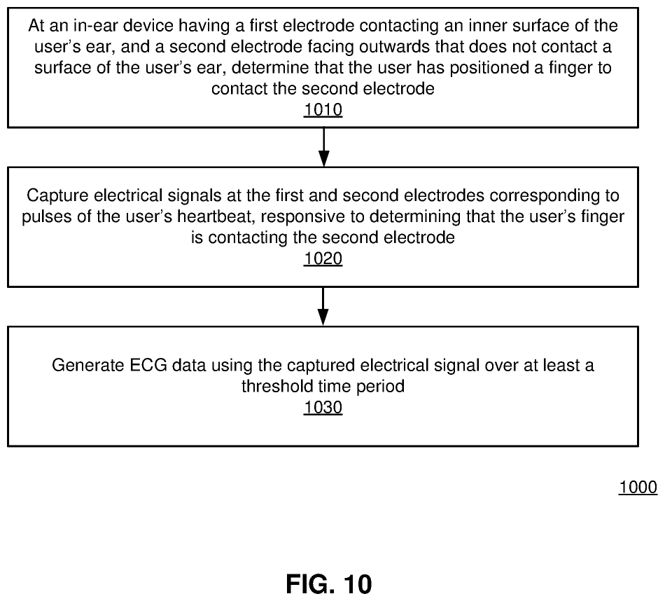

FIG. 10 is a flowchart of a method 900 for measuring an ECG of a user, in accordance with one or more embodiments. The process shown in FIG. 10 may be performed by components of an ECG monitoring system (e.g., ECG monitoring system 100) to monitor a user's cardiac activity, the ECG monitoring system including an in-ear device positioned in a user's ear to have a first electrode contacting an inner surface of the user's ear, and a second electrode facing outwards that does not contact a surface of the user's ear. As such, the first electrode corresponds to an in-ear electrode, and the second electrode corresponds to an out-of-ear electrode. Other entities may perform some or all of the steps in FIG. 10 in other embodiments. Embodiments may include different and/or additional steps, or perform the steps in different orders.

The in-ear device (e.g., a processor of the in-ear device, such as a signal processor) determines 1010 that the user has positioned a finger to contact the second electrode. In some embodiment, the signal processor infers that the user has positioned a finger to contact the second electrode based upon a voltage measurement of the second electrode. In some embodiments, the determination that the user has positioned a finger to contact the second electrode may be performed by a processor of a monitoring device external to the in-ear device (e.g., by a processor of a headset worn by the user). In some embodiments, the in-ear device may prompt the user to place a finger on the second electrode, e.g., as an audio prompt using an audio transducer, and determining that the user has positioned their finger on the second electrode based upon a measured voltage, pressure, etc. In some embodiments, the in-ear device may prompt the user to place a specific finger (e.g., index finger) on the second electrode, to ensure consistent ECG measurement. In some embodiments, the in-ear device may perform voltage or electrical impedance monitoring to detect when the user has contacted the second electrode with their finger. In other embodiments, an optical proximity sensor (e.g., a photodetector, or a pair of LEDs and a photodetector) may be used to detect when the user is touching the second electrode with their hand (e.g., due to a detected light change).

Responsive to determining that the user's finger is contacting the second electrode, the in-ear device captures 1020 electrical signals at the first and second electrodes corresponding to pulses of the user's heartbeat. In some embodiments, the in-ear device may instruct the user (e.g., via an audio prompt) to maintain contact with the second electrode for at least the threshold period of time, and/or notify the user when a threshold period of time has elapsed.

A monitoring device generates 1030 ECG data using the captured electrical signals over at least the threshold time period. In some embodiments, the monitoring device comprises a processor external to the in-ear device that is in communication with the in-ear device. In other embodiments, some or all the functions of the monitoring device are performed by a processor within the in-ear device. In some embodiments, the generated ECG data corresponds to a voltage difference between the first and second electrodes. In some embodiments, the ECG data is generated based upon an amplified voltage difference between the first and second electrodes generated using a differential amplifier of the signal processor of the in-ear device. In some embodiments, the generated ECG data is analyzed (e.g., by a processor of the in-ear device or by an external processor) to determine one or more additional user health metrics, such as a user heartbeat rate, a user blood pressure level, etc.

The method 1000 may be repeated to continuously monitor ECG data of the user over time. In some embodiments, different configurations may be used to capture ECG data along different viewing vectors, based on which an overall ECG profile for the user may be constructed. In some embodiments the in-ear device may prompt the user to use a certain hand to touch the second electrode, or use both hands to touch the second electrodes of respective in-ear devices worn in each ear of the user, to generate ECG data along a particular viewing vector. In some embodiments, the in-ear device may infer a viewing vector of measured ECG data based upon a polarity of the measured ECG data. In some embodiments, different types of sensors may be used to capture different sets of ECG data of the user (e.g., the in-ear device in out-of-clinic settings, and a hospital-grade ECG monitoring system for in-clinic settings), the results of which may be combined to determine an overall picture of the user's cardiac health.

Through the use of in-ear devices having one or more in-ear electrodes, the user is able to take ECG measurements easily in an out-of-clinic setting. For example, as discussed above, using a single in-ear device with at least one in-ear electrode and at least one out-of-ear electrode, bipolar ECG data equivalent to the use of a head electrode and an arm electrode can be measured. In addition, by using different hands, the user is able to measure ECG data along different viewing vectors, affording a more complete picture of the user's cardiac activity and health.

In addition, because the user's ear is generally a stable location on the user's body, measuring ECG data through an in-ear electrode on the in-ear device may yield a clearer signal with fewer artifacts in comparison to other body parts such as the user's wrist. In addition, in some embodiments, the in-ear device may be comfortable enough to be worn all-day by the user, allowing the user to monitor their health and take ECG measurements on the fly. This allows the user to measure their cardiac activity and obtain an ECG in various settings whenever desired.

While the above figures discuss the ECG monitoring system primarily in the context of an in-ear device, it is understood that in other embodiments, other types of devices may be used to for ECG monitoring. For example, on a headset such as that described in FIG. 2A, first electrodes contacting the user's head may be located on a nose pad of the headset, near the user's temples, or near the user's pinna, while second world-facing electrodes that do not contact the user's head may be located on the frame of the headset. The user may initiate ECG measurements by touching a second world-facing electrode with their fingertip, in a manner similar as described above.

In some embodiments, the techniques described herein can also be used for other electrophysiological sensing such as electroencephalography (EEG) applications for attention tracking, etc. For example, in some embodiments, the in-ear electrodes of the in-ear device may be used as electrodes as part of an EEG application. In some embodiments, the user may initiate measurement of EEG data by touching the out-of-ear electrode with their fingertips, signaling to a processor (e.g., signal processor) of the in-ear device to perform EEG measurements.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A system, comprising:
an in-ear device comprising:
at least one in-ear electrode configured to contact an inner surface of a user's ear when the in-ear device is worn by the user;
an out-of-ear electrode configured to not contact a surface of the ear of the user when the in-ear device is worn by the user; and
a processor configured to:
determine that a finger of the user is contacting the out-of-ear electrode;
responsive to determining that the finger is contacting the out-of-ear electrode, capture electrical signals corresponding to a heartbeat of the user at the at least one in-ear electrode and the out-of-ear electrode; and
generate electrocardiogram (ECG) data based upon the captured electrical signals.

2. The system of claim 1, wherein the out-of-ear electrode is located on an outer surface of the in-ear device and configured to face away from the user's head when the in-ear device is worn by the user.

3. The system of claim 1, wherein the processor is configured to capture electrical signals corresponding to the heartbeat at the at least one in-ear electrode and the out-of-ear electrode over at least a threshold period of time.

4. The system of claim 1, wherein the processor is further configured to determine a heart rate of the user based upon the generated ECG data.

5. The system of claim 1, wherein the generated ECG data comprises first ECG data generated by the processor during a first time period, and wherein processor is further configured to generate second ECG data based upon captured electrical signals at the at least one in-ear electrode and the out-of-ear electrode during a second time period, responsive to determining that that the user is contacting the out-of-ear electrode using a finger of a second hand of the user.

6. The system of claim 1, wherein the in-ear device further comprises an audio transducer configured to provide audio messages to the user.

7. The system of claim 6, wherein the audio transducer is configured to prompt the user to contact the out-of-ear electrode with the finger.

8. The system of claim 1, wherein the at least one in-ear electrode comprises a plurality of in-ear electrodes configured to contact different locations of the inner surface of the ear of the user when the in-ear device is worn by the user, and wherein the processor is further configured to aggregate electrical signals corresponding to the heartbeat of the user captured at each of the plurality of in-ear electrodes.

9. The system of claim 1, wherein at least a portion of the processor is located in the in-ear device.

10. The system of claim 1, wherein the processor is located in a headset.

11. The system of claim 1, wherein the in-ear device further comprises a signal processor configured to amplify a voltage difference between the electrical signals captured at the at least one in-ear electrode and at the out-of-ear electrode.

12. The system of claim 1, further comprising:
a second in-ear device comprising at least one in-ear electrode and an out-of-ear electrode, and
wherein the processor is further configured to:
responsive to determining that the user is contacting the out-of-ear electrode of the in-ear device with a first finger, and the out-of-ear electrode of the second in-ear device with a second finger, capture second electrical signals corresponding to the heartbeat of the user at the the out-of-ear electrode of the in-ear device and at the out-of-ear electrode of the second in-ear device; and
generate second electrocardiogram (ECG) data based upon the captured second electrical signals.

13. A method, comprising:
at an in-ear device comprising at least one in-ear electrode configured to contact an inner surface of a user's ear when the in-ear device is worn by the user and an out-of-ear electrode configured to not contact a surface of the ear of the user when the in-ear device is worn by the user, determining that a finger of the user is contacting the out-of-ear electrode;
responsive to determining that the finger of the user is contacting the out-of-ear electrode, capturing electrical signals corresponding to a heartbeat of the user at the at least one in-ear electrode and the out-of-ear electrode; and
generating electrocardiogram (ECG) data based upon the captured electrical signals.

14. The method of claim 13, wherein the out-of-ear electrode is located on an outer surface of the in-ear device and configured to face away from the user's head when the in-ear device is worn by the user.

15. The method of claim 13, further comprising capturing electrical signals corresponding to the heartbeat of the user at the at least one in-ear electrode and the out-of-ear electrode over at least a threshold period of time.

16. The method of claim 13, further comprising determining a heart rate of the user based upon the generated ECG data.

17. The method of claim 13, wherein the generated ECG data comprises first ECG data generated during a first time period, and further comprising generating second ECG data based upon captured electrical signals at the at least one in-ear electrode and the out-of-ear electrode during a second time period, responsive to determining that that the user is contacting the out-of-ear electrode using a finger of a second hand of the user.

18. The method of claim 13, wherein the at least one in-ear electrode comprises a plurality of in-ear electrodes configured to contact different locations of the inner surface of the ear of the user when the in-ear device is worn by the user, and wherein the method further comprises aggregating electrical signals corresponding to the heartbeat of the user captured at each of the plurality of in-ear electrodes.

19. The method of claim 13, further comprising amplifying a voltage difference between the electrical signals captured at the at least one in-ear electrode and at the out-of-ear electrode.

20. A headset, comprising:
at least a first electrode configured to contact a surface of a user's head when the headset is worn by the user;
a second electrode configured to not contact a surface of the user's head when the headset is worn by the user; and
a processor configured to:
determine that a finger of the user is contacting the second electrode;
responsive to determining that the finger is contacting the second electrode, capture electrical signals corresponding to a heartbeat of the user at the at least one first electrode and the second electrode; and
generate electrocardiogram (ECG) data based upon the captured electrical signals.
